(19) United States
(12) Patent Application Publication
(10) Pub. No.: US 2008/0050309 A1
(43) Pub. Date: Feb. 28, 2008

(54) Compositions of pharmaceuticals for use in low energy neutron therapy

(75) Inventors: Zbigniew Janusz Witczak, Mountain Top, PA (US); Frederick William Fischer, Solebury, PA (US)

Correspondence Address: Zbigniew Janusz Witczak, 520 Robins Way, Mountain Top, PA, 18707, US

(73) Assignee: Hadron Systems, Solebury, PA (US)

(21) Appl. No.: 11/509,423

(22) Filed: Aug. 25, 2006

(51) Int. Cl. A61K 51/00; C07F 5/00

(52) U.S. Cl. 424/1.11; 534/15

(57) ABSTRACT

A pharmaceutical, wherein the formulation is a conjugate of a saccharide with one or a plurality of isotopic boron-10 atoms or gadolinium, and having a utility in the binary form of low energy neutron therapy (i.e. boron neutron capture therapy). Further, that the therapy has an indicated use for the selective targeting and killing of oxygenated and hypoxic cells in tumors, and that the pharmaceutical is preferentially taken up by cancer cells. The inclusion of any of a family of saccharides, its derivatives or analogues, including sulfur linkage saccharides as a delivery carrier for boron-10 atoms or the rare element gadolinium is a strategy to take advantage of the hallmark of cancer cells, that being an increased requisite for glucose to sustain a rate of uncontrollable cell division and proliferation. A cancer cell facilitates an increase in availability of molecular glucose by overexpression of glucose transporter proteins on membrane surfaces. The use of a saccharide attached with one or more of isotopic boron-10 atoms or gadolinium is to utilize this known amplification of glucose transport to achieve significantly greater quantities of isotopic boron-10 atoms or gadolinium taken up cancer cells than deposited into healthy cells.

Tissues of malignant tumors are not homogeneous, but consist of oxygenated and hypoxic cells. Oxygen deprivation causes cancer cells to be resistant to radiation and to chemotherapeutic agents. It is further noted that the etiology of metastasis is hypoxia induced tumor cells, where oxygen deprivation stimulates activation of genes, and that one gene is responsible for programming oxygen-starved cancer cells to migrate to distant and specific host organs.

A saccharide with a ring sulfur atom can participate more readily in biological processes than a sugar with a ring oxygen atom, and such thiosaccharides possess unique physiochemical properties that include penetration of a viscous lipid bilayer membrane of a cancer cell, and resistance to reduction by enzymes, enabling retention of the conjugate within cytoplasm, mitochondria and nuclei of cancer cells. Therefore, an objective is the use of a conjugated boron-10 or gadolinium thiosaccharide as a pharmaceutical for low energy neutron therapy to participate in a boron-10 interception of a passing slow (thermal) neutron to produce an α-particle, high energy Li-7 ion and low energy gamma (γ) rays that are damaging to a cell. The pharmaceutical, when in combination with exposure of a subject to low energy neutrons, is a method of treatment for a subject diagnosed with a cancer, so as to cause a regression of tumors, to inhibit metastasis, and to extend life.

### CROSS REFERENCE TO RELATED APPLICATIONS

This application gains priority from provisional patent application Ser. No. 60/596,001 filed Aug. 23, 2005, the provisional application being herein incorporated by reference.

### TECHNICAL FIELD AND BACKGROUND ART

The present invention relates to a binary form of low-energy neutron therapy (i.e. boron neutron capture therapy) in which a pharmaceutical, containing isotopic boron-10 or the rare element gadolinium attached to a carrier molecule, is administered to a cancer subject, followed by exposure of the subject to a stream of low energy neutrons. The cross section of isotopic boron-10 for neutrons is greater than three orders of magnitude higher than for other nuclei common to living tissue (such as hydrogen, oxygen, carbon), allowing for cells containing boron-10 atoms to be dosed with neutrons at a sizable flux, yet have a minimal effect on boron-free cells in the beam path. As low energy (thermal) neutrons pass through a cell where there is the presence of isotopic boron atoms, an atom will intercept the slow moving neutron (<5 km/sec). The interception of a passing neutron by a boron-10 atom leads into a fissionable reaction, with excitation of the boron atom, producing an unstable boron-11 for a brief period, and splitting of the atom. The reaction releases two highly energetic particles, a He⁴ ion (alpha particle) and Li⁷ ion. In almost all boron neutron captures, a 480 keV gamma particle is emitted in addition to the alpha and lithium particles. The combined linear distance traveled by both particles is <10 microns, limiting the effect of the reaction to single cells. The required quantity of neutron boron-10 reactions to elicit a lethal effect is generally estimated at 10⁹ B-10 atoms in a cell, which translates to approximately 35 μg per gram of tissue, to result in breaks to helix double strand DNA.

The highly damaging effect resulting from the neutron and boron-10 interaction makes it essential that a pharmaceutical possess selectivity properties for malignant cells as to minimize collateral damage to normal cells and tissue. For the pharmaceutical to produce a desirable outcome in low energy neutron therapy, a pharmaceutical must demonstrate preferential uptake by cancer cells vs. normal cells, a functionality for transport across the viscous lipid bilayer of the cancer cell membrane, localization in cytoplasm and the nucleus of a cancer cell, and retention through resistance to enzymatic reduction.

Numerous boron-10 containing compounds using a diversity of carrier molecules have been synthesized with the objective to deliver isotopic boron-10 to cancer cells. Related art includes: a composition of a conjugate of boron-10 to at least one antibody or antigen-binding antibody fragment, which selectively binds to an antigen on the cell surface produced by or associated with the tumor cells; nucleosides and oligonucleosides containing boron clusters; P-boronophenylalanine complexes with fructose and related carbohydrates and poltols; a sodium borocaptate (sulfhydryl boron hydride); porphyrins, including sapphyrins and texaphyrins containing multiple carborane cages; and other compounds, including liposomes. Only one compound is currently in use as a pharmaceutical agent in investigational studies of boron neutron capture therapy for the treatment of brain cancers: boronated phenylalanine, and boronated phenylalanine with fructose.

Pharmaceuticals that are in use and pharmaceuticals shown in prior art do not completely satisfy the application of boron neutron capture therapy. Phenylalanines, sub-classes of amino acids, are taken up by cells in minute amounts, and of such inconsequential quantities to be meaningless to the number of boron-10 atoms intercepting low-energy neutrons required to elicit an effect within a cancer cell. A protocol exists for boronated phenylalanine that is co-administered with fructose. This is the administration, via infusion, of two distinct and separate substances, not a conjugate of boronated phenylalanine to fructose (fructose is transported into cells via GLUT-4), and it remains unclear how the administration and absorption of fructose benefits the accompanying boronated phenylalanine. Sodium borocaptate and porphyrinated compounds display only a slight affinity for neoplastic cells and tumor cells, and are not in use do to a high degree of toxicity. Monoclonal antibodies bind to antigens on the cell surface, yet interception of low-energy neutrons by boron-10 at the cell surface contradicts the concept of BNCT, where the fissionable reaction is to occur within the cancer cell. Released highly-energetic alpha particles and lithium ions travel in a linear pathway, and for the fissionable reaction to occur on the cell surface means the probability of only one, and possibly neither, highly energetic particle passing through the cancer cell, or resulting in any sustainable damage to the cancer cell.

As malignant cells rapidly proliferate and form a tumor, there is a heightened requisite for energy to sustain the rate of uncontrolled abnormal growth. Angiogensis is induced to deliver blood containing glucose, nutrients, other substrates, and oxygen to the growing tumor. The formation of new blood vessels typically fails to keep pace with the growth of tumors, limiting the supply of blood to cells proximate to the surface of a growing tumor, and resulting with regions of the tumor where cells become starved of nutrients and of molecular oxygen. This lack of homogeneous distribution of oxygen and nutrients in a tumor leads to the occurrence of hypoxia in cells. It is known that oxygen starvation in tumor cells makes cells resistant to the lethal effects of ionization in radiotherapy treatment regimens. It is also evident that hypoxia in cancer cells is the dominant factor in resistance by those cells to the majority of chemotherapeutic agents. The occurrence of hypoxia in cells is limited to tumors. Completion of a treatment regimen of radiotherapy or of chemotherapy may result in effectively killing nearly all oxygenated cancer cells at and near the periphery of a tumor, but failure of a cancer treatment to eradicate an inner stratum of oxygen depleted cells leaves a viable and aggressive malignant tumor and, consequently, a cancer that is more difficult to treat.

The ability of primary tumors to metastasize is the dominant reason for cancer having a high rate of mortality. The condition of hypoxia in malignant cells occurs in a region of a tumor where there is no vascularity. The absence of a continuous blood supply to tumor cells means deprivation of molecular oxygen, which initiates tumor cells to respond by inducing changes to biochemical pathways. Under normal oxygenation of cancer cells, a gene (von Hippel-Lindau [pVHL]) modifies the α-subunits of hypoxia-inducible factor (HIF) for degradation. In the absence of availability of molecular oxygen, this suppressive action cannot occur, and levels of oxygen-deficit related protein HIF increase. Accumulation of SIF activates the expression of numerous hypoxia-inducible genes; two of which stimulate synthesis of erythropoietin and of the outgrowth of blood vessels, two other identified genes (c-MET and CXCR4) program malignant tumor cells to migrate, and to home in on specific, distant organs. This over-expression of HIF and activation of the CXCR4 gene plays a key role in the process of cell dissemination and the metastasizing of tumors.

### SUMMARY OF THE INVENTION

In a first embodiment of the invention, there is provided a pharmaceutical that is a composition of isotopic boron-10 or gadolinium attached to a delivery carrier. In a further embodiment of the invention, there is provided a pharmaceutical that where the delivery carrier is given as either a monosaccharide or a disaccharide, derivatives of said saccharides and of analogues. The invention includes a conjugate of isotopic boron-10 or gadolinium to a monothiosaccharide or dithiosaccharide, both sugars having a sulfur atom instead of oxygen atom in the ring or a sulfur linkage via a bridge. The advantages of a sulfur atom in the ring, as opposed to an oxygen atom, have been postulated over the years, particularly with regard to C—S—C bond distances obtained with sulfur. C—S—C bonds create different angles and conformation, where the C—S bond is longer (ca 1.8A) and the C—S—C angle (ca. 95-100°) is smaller than the corresponding oxygen-containing structure. Moreover, the stronger electronegativity of the sulfur atom in comparison to oxygen contributes to excellent stability of the S-linkages, and the water solubility of sulfur derivatives is generally higher than their oxygen counterparts, an important advantage over the chemical characteristic of oligosaccharides.

In accordance with the objects of the invention, a method for preferentially targeting cancer cells over normal cells is provided. All cells depend on a continuous supply of glucose, which is a major substrate for energy production, but the rate of metabolism is acutely accelerated in cancer cells. Malignant cells respond to a heightened demand for glucose through consequential changes to cancer cell membrane surfaces with the overexpression of a family of facilitative glucose transporter (GLUT) proteins. The increase in cell surface transporters facilitate the additional uptake of glucose to meet the energy needs to sustain a process of uncontrollable cell division and proliferation. GLUT is present on the surfaces of all normal cells and of all cell types, but this overexpression of GLUT on cancer cell membrane surfaces is the specific differentiation by which the pharmaceutical is taken up by cancer cells at a higher rate than by normal cells.

The invention includes the unique physiochemical properties of thiosaccharides for delivery and transport of the pharmaceutical, containing gadolinium or isotopic boron-10 atoms, into hypoxic cells, which are resistant to ionizing radiation and to most chemotherapeutic agents. Additionally, oxygen starved cancer cells allow for accumulation of hypoxia-inducible factor (HIF) inside of cells, and HIF stimulates the outgrowth of new blood vessels and programs hypoxic cells to migrate away from oxygen depleted tissues, instigating the process of a metastasis.

In another embodiment of the invention, the pharmaceutical is used in combination with a stream of slow (i.e. epithermal, thermal or cold) neutrons in a binary form of low-energy neutron therapy (as in boron neutron capture therapy). Physics of the therapy relate to the interception of neutrons by Boron-10 atoms, leading to the reaction initiated by creating an unstable boron-11 that instantly splits to yield a helium-4 nucleus (alpha particle) and a lithium-7 nucleus. In the majority of reactions, 480 keV of gamma radiation is emitted in addition to the helium and lithium particles. The released high energy linear (LET) alpha and lithium particles travel a linear distance of <10 microns, which is the approximate size of a single cell; thus the damaging or lethal effect is confined to single cells.

An object of the invention is localization and retention of the boron-10 atoms or gadolinium in cytoplasm, mitochondria and nuclei in cancer cells. A further objective of the invention is the transport of a sufficient quantity of the pharmaceutical into cancer cells that allow for the number of boron neutron captures and released He-4 and Li-7 nuclei to reach a lethal effect. Basic calculations show that the occurrence of 103 reactions in a cell result in a total absorbed dose of 2.31 MeV radiation, which translates to a lethal dose of radiation.

It is therefore an object of the present invention to provide a method for the treatment of cancer using a pharmaceutical, the chemistry being the covalent attachment of one or more isotopic boron-10 atoms, a cluster of boron-10 atoms, or gadolinium to a carrier molecule, and that the pharmaceutical is to be used in a binary form of low-energy neutron therapy with the indicated use for the treatment of cancer.

The method for use of the pharmaceutical involves administration to a subject, and that the pharmaceutical is in a solution for administration via intravenous route, or administration of the solution directly into a tumor, or administration of the solution into a lymph node, or any combination of routes of administration of a solution.

### DETAILED DESCRIPTION OF SPECIFIC EMBODIMENTS

The invention is of a pharmaceutical used in combination with streaming slow neutrons in the binary method of a low energy neutron therapy for the treatment of cancer. The pharmaceutical is the attachment of an isotopic boron-10 atom or gadolinium to one of a series of saccharides, a saccharide derivative or an analogue of a saccharide, or the attachment of a plurality of isotopic boron-10 atoms at different positions of one of a series of saccharides, or the attachment of a cluster of isotopic boron-10 atoms to one of a series of saccharides.

In further accordance with the invention, the pharmaceutical is the attachment of an isotopic boron-10 atom or gadolinium to one of a series of thiosaccharides, a thiosaccharide derivative, or an analogue of a thiosaccharide, or the attachment of a plurality of isotopic boron-10 atoms at different positions of one of a series of thiosaccharides, or the attachment of a cluster of isotopic boron-10 atoms to one of a series of thiosaccharides.

A saccharide containing a sulfur atom in a C—S—C linkage or a disaccharide linked via a sulfur bridge, as opposed to other linkage atoms such as oxygen, is shown to have specific advantageous biological properties, including solubility, which differs from conventional monosaccharides. Other advantages of thiosaccharides, pertinent to biological involvement, include conformational, density, and flexibility differences, while other distinct differences of thiosugars from oxygen-linkage sugars are less electronegativity and greater polarity. Moreover, thiosaccharides have a specific affinity to lipid layers of cell membranes, characteristic of malignant cells, through the selective inhibition of fatty acid synthetase. The cytoplasm of cancer cells is a highly reducing environment in which protein cysteines reduce glucose to products used in the metabolic pathway. The thiol (—SH) or thiolate (—S—) state creates a unique resistance to metabolizing enzymes (hydrolases) of a cell, thereby stabilizing the conjugated boron-10 or gadolinium thiosaccharide analogue and achieving retention in the cell, essential to low-energy neutron therapy.

Malignant tumors contain hypoxia-induced cells. Cells starved of molecular oxygen undergo changes in molecular pathways and activation of the gene, CXCR4, which programs hypoxia-inducible metastasis, significantly increasing the morbidity of the disease. A unique property of a saccharide having sulfur linkage is transport into hypoxic cells, in contrast to sugars having oxygen linkages that fail to achieve uptake by hypoxic cells.

The method for use of the pharmaceutical involves a stream of neutrons, decellerated to low-energy via a moderator. As the velocity of a neutron slows, the wave length of the neutron dramatically increases. The correspondent to the increased wave length of a neutron is the boron-10 nucleus, which has a large neutron absorption cross section for slow neutrons. A boron-10 atom absorbs slow neutrons at a magnitude several thousand times greater than of the elements constituting living tissue (hydrogen, oxygen, carbon, etc). By selectively depositing isotopic boron-10 atoms in cancer cells, while sparing normal cells, low energy neutron therapy becomes a highly effective method for the eradication of tumors, infiltrative tumors, precursors of tumors, and migrating cancer cells.

The therapy is performed by administering the pharmaceutical in solution by injection via an intravenous route, by catheter, an injection directly into a tumor, an injection directly into a lymph node, or a combination of routes of administration. The therapy calls for a period of time between the administration and exposure to slow neutrons. This phase allows for the pharmaceutical to be taken up preferentially by cancer cells and retained in cytoplasm, mitochondria and nuclei of cancer cells. The phasing between administration of the pharmaceutical and exposure to a stream of low-energy neutrons allows for maximizing the concentration of isotopic boron-10 or gadolinium in cancer cells when the subject is treated with neutrons.

What is claimed is:

1. A pharmaceutical compound having a composition of one or a plurality of isotopic boron-10 atoms or of gadolinium attached to a saccharide as the carrier molecule
where the carrier molecule is a monosaccharide, a monosaccharide derivative or an analogue of a monosaccharide
where the carrier molecule is a disaccharide, a disaccharide derivative, or an analogue of a disaccharide

2. A pharmaceutical compound having a composition of one or a plurality of isotopic boron-10 atoms or of gadolinium attached to a thiosaccharide as the carrier molecule
where a thiosaccharide has a sulfur atom replacing an oxygen atom in the ring
where the C—S bond is longer (1.8 Å vs. 1.43 Å) and the C—S—C angle is smaller (96-100° vs. 110-113°) than the corresponding oxygen-containing structure
where the carrier molecule is a monothiosaccharide, a monothiosaccharide derivative or an analogue of a monothiosaccharide
where the carrier molecule is a dithiosaccharide, a dithiosaccharide derivative or an analogue of a dithiosaccharide.

3. A pharmaceutical compound according to claim 2, where a sulfur linkage saccharide possesses unique properties in electron density and solubility, enabling the pharmaceutical to participate more readily in biological processes

4. A pharmaceutical compound according to claim 2, where a sulfur atom replacing an oxygen atom in the ring endows the molecule with properties facilitating transport through portals of viscous membranes of cancer cells.

5. A pharmaceutical compound according to claim 2, where a sulfur atom replacing an oxygen atom in the ring endows the molecule with properties to resist enzymes and processes of reduction or transformation.

6. A pharmaceutical compound according to claims 1, 2, wherein the conjugate of isotopic boron-10 atoms or gadolinium and a carrier molecule binds to any within a family of facilitative glucose transporters (GLUT) proteins expressed on cell membrane surfaces.
where the method for targeting cancer cells is based on the overexpression of GLUT on cancer cell membrane surfaces.

7. A pharmaceutical compound according to claim 2, that is taken up by hypoxic tumor cells.

8. A pharmaceutical compound according to claims 1, 2 that is in a solution composition for administration via an intravenous route, or via injection directly into a tumor, or via injection directly into a node of the lymphatic system.

9. A pharmaceutical compound according to claims 1, 2 that is used in the method of a binary low energy neutron therapy, combining administration of the pharmaceutical followed by exposure of the subject to streaming low energy (thermal) neutrons as a non-invasive treatment for cancer
(a) where the proliferative disease is a primary malignant tumor,
(b) where the proliferative disease is an infiltrative tumor,
(c) where the proliferative disease is neoplastic tissue,
(d) where the proliferative disease is metastasis,
(e) and where the proliferative disease is disseminated malignant cells infiltrating blood vessels, the lymphatic system, or nerves of the central nervous system.
